(19) United States
(12) Patent Application Publication
(10) Pub. No.: US 2025/0061550 A1
(43) Pub. Date: Feb. 20, 2025

(54) ULTRASOUND IMAGE ARTIFACT REDUCTION

(71) Applicant: SINTEF TTO AS, Trondheim (NO)

(72) Inventors: Rune Hansen, Trondheim (NO); Jørgen Avdal, Trondheim (NO)

(21) Appl. No.: 18/719,941

(22) PCT Filed: Dec. 14, 2022
(86) PCT No.: PCT/EP2022/085969

§ 371 Date: Jun. 14, 2024

(30) Foreign Application Priority Data
Dec. 14, 2021 (GB) 2118089.8

(51) Int. Cl. G06T 5/70; G01S 7/52; G06T 5/10; G06T 7/00; G06T 7/11

(52) U.S. Cl. CPC G06T 5/70; G01S 7/52028; G01S 7/52077; G06T 5/10; G06T 7/0012; G06T 7/11; G06T 2207/10132; G06T 2207/20064; G06T 2207/30004

(57) ABSTRACT

There is provided a method for suppressing an image artifact in an ultrasound data set of an anatomical structure, the anatomical structure comprising a volume of biological tissue having a first acoustic attenuation and a fluid-filled cavity having a second acoustic attenuation lower than the first acoustic attenuation. The method comprises obtaining a first ultrasound data set of the anatomical structure including the biological tissue and a fluid-filled cavity, the first ultrasound data set comprising a region of interest; processing the first ultrasound data set using computer implemented signal or image processing techniques, wherein the processing includes compensating for the lower acoustic attenuation in the fluid-filled cavity; and obtaining a second ultrasound data set of the anatomical structure including the biological tissue and the fluid-filled cavity, wherein the second ultrasound data set comprises said region of interest and where image artifacts related to the lower acoustic attenuation of said fluid-filled cavity are suppressed relative to said first ultrasound data set.

The present invention relates to a method and a system for suppressing ultrasound image artifacts related to fluid-filled cavities.

Ultrasound is widely used for imaging anatomical structures and tissues for medical purposes due to its relatively low cost and ease of use. One particular application of diagnostic ultrasound is intraoperative ultrasound imaging in neurosurgery. This application is used as an example for describing the present invention but the invention is relevant for all situations involving imaging in relation to fluid-filled cavities and the invention is hence not to be limited by the specific application of intraoperative ultrasound imaging in neurosurgery.

Whilst most cancer types have seen a significant improvement in treatment and survival, median survival for common brain tumors is still only about 15 months, even with the most advanced and up-to-date treatment strategies. For some types of brain tumor, e.g., diffuse gliomas, the initial optimal treatment is surgery. Maximal surgical resection of the tumor is associated with improved survival for both high-grade and low-grade gliomas. However, it can be challenging to obtain maximal resection of the tumor whilst avoiding damaging healthy brain tissue. The maximal safe resection of low-grade gliomas is particularly challenging because the tumor is nearly impossible to distinguish from normal brain tissue in a surgical microscope.

One commonly used tool to guide tumor resection is the conventional navigation system in which pre-operative magnetic resonance (MR) images of the patient are mapped to the patient on the operating table. The system then works as a “surgical GPS”, enabling the surgeon to identify a spatial location on the patient with a tracked instrument and see the corresponding location in the MR images. This system is useful for optimal placement of the surgical opening and finding the tumor. However, as the resection progresses the pre-operative MR images are outdated and do not provide information about the current status regarding the remaining tumor tissue and distance to critical structures such as major blood vessels and white matter tracts (for brain tumors). In order to provide updated information during surgery, intraoperative imaging can be performed to follow the progress of the tumor resection.

Intraoperative magnetic resonance imaging (iMRI) can provide images during surgery. However, the high cost combined with the need for specially adapted operating rooms severely limits the use of this solution. In addition, the time requirements for imaging often limit the use to a single scan towards the end of the procedure.

Intraoperative ultrasound imaging presents a number of advantages in this respect. Ultrasound scanners are typically portable, relatively cheap and readily available in most surgical departments. In addition, ultrasound offers real-time imaging and imaging can be performed repeatedly to follow the progress of the operation, e.g., of the tumor resection. During resection surgery, the ultrasound probe can be tracked by the navigation system and the ultrasound images can then be displayed together with the pre-operative MR images. The ultrasound images can therefore be used to guide the tumor resection. To optimally guide this procedure, high quality images are required.

One factor which can decrease the quality of ultrasound images is the presence of image artifacts. These are misrepresentations of the tissue structures produced when the assumptions inherent to the applied imaging techniques are not adequately valid. In ultrasound imaging for tumor resection procedures, image artifacts can occur due to the formation of a surgical cavity as the tumor is resected. The cavity is typically filled with saline to provide the required acoustic contact between the ultrasound probe and the tissue being imaged. However, the acoustic attenuation for saline is negligible compared to soft tissues, which can cause the part of the image behind the cavity to be excessively bright due to low attenuation of the ultrasound signals through the saline. That is, the received ultrasound echoes from remaining tumor tissue can be excessively strong due to relatively low attenuation in the propagation path between the probe and the tumor through the saline. This significantly reduces the contrast resolution in the image and hence the ability to differentiate normal tissue and tumor tissue and this artifact can therefore result in suboptimal removal of tumor mass.

The described artifact, arising due to low acoustic attenuation of a fluid-filled cavity, can also arise from anatomical structures having naturally-occurring fluid-filled cavities, such as for example large blood vessels, the bladder, the gall bladder, the heart, cysts and an amniotic sac. The resulting artifact can then result in misinterpretation, inter-examiner variability and reduced diagnostic accuracy.

For diagnosis of cysts, the indicated artifact is used to differentiate cysts from hypoechoic solid lesions and the indicated artifact can in this situation therefore be useful. However, for evaluation of tissue structures occurring behind a cyst, the possibility to strongly suppress the indicated artifact can be of interest. Hence, by displaying both an original ultrasound image with the artifact and a processed ultrasound image with artifact suppression, the cyst can be diagnosed by the artifact and the tissue structures occurring behind the cyst can be better evaluated.

For evaluation of atherosclerotic plaques, the plaque echogenicity is sometimes used as an indicator of plaque vulnerability and risk of rupture. Relative to the ultrasound probe, atherosclerotic plaques can be situated at the proximal wall of a blood vessel or at the distal wall of a blood vessel. For frequencies used in transcutaneous ultrasound imaging (i.e., below 20 MHz), blood has significantly lower acoustic attenuation relative to soft tissues. For imaging of atherosclerotic plaques situated at the distal wall of a blood vessel, the ultrasound waves must travel through the low-attenuating blood in the lumen of the blood vessel before reaching the atherosclerotic plaque. The described artifact will therefore be present albeit not as pronounced as for a resection cavity containing saline because the acoustic attenuation for blood is higher than for saline. The artifact will, however, to some extent affect the echogenicity of a plaque situated at the distal wall of a blood vessel and suppressing the artifact can be of interest.

In resection procedures, one solution that has been proposed is to replace the saline in the resection cavity with a synthetic liquid having an acoustic attenuation similar to brain tissue (Selbekk, T., et al., Acta neurochirurgica, 2013). However, the use of a synthetic liquid is invasive and poses a potential hazard to the patient. Additionally, exposure of the cavity to a synthetic liquid will require some sort of cavity cleaning after use which will prolong the surgical intervention, and adequate cleaning of such a cavity can be challenging.

Herein, an ultrasound data set is defined as a set of data acquired from an ultrasound imaging device, including any new data set produced from said acquired data set using any type of processing, including the final images or post-processed images. By ultrasound image we mean any visual display of the ultrasound data set.

Viewed from a first aspect, the invention provides a method for suppressing image artifacts in an ultrasound data set of an anatomical structure, the anatomical structure comprising a volume of biological tissue having a first acoustic attenuation and a fluid-filled cavity having a second acoustic attenuation lower than the first acoustic attenuation, the method comprising: obtaining a first ultrasound data set of the anatomical structure including the biological tissue and the fluid-filled cavity, the first ultrasound data set comprising a region of interest; processing the first ultrasound data set using computer implemented signal or image processing techniques, wherein the processing includes compensating for the lower acoustic attenuation in the fluid-filled cavity; and obtaining a second ultrasound data set of the anatomical structure including the biological tissue and the fluid-filled cavity, wherein the second ultrasound data set comprises said region of interest and where image artifacts related to the lower acoustic attenuation of said fluid-filled cavity are suppressed relative to said first ultrasound data set.

The present method thus post-processes the first ultrasound data set to compensate for the low attenuation region of said fluid-filled cavity using computer implemented signal or image processing, allowing a new (second) ultrasound data set to be obtained in which the low attenuation cavity region has been compensated for.

The anatomical structure may comprise a brain, a bladder, a gall bladder, a fetus, a heart, an atherosclerotic plaque in an artery wall, or any other anatomical structure in relation to naturally-occurring or surgically created (e.g., via tissue resection) fluid-filled cavities.

The obtaining of the second ultrasound data set is preferably based on the processing of the first ultrasound data set, so that the cavity region is accurately compensated for within the second ultrasound data set.

In some examples, the second ultrasound data set may be obtained by modifying, e.g., attenuating, the first ultrasound data set. Said attenuation preferably comprises applying a filter to at least part of the data set, e.g., to the region of interest. In such examples the attenuation may be understood as “artificial attenuation” because the computer software simulates attenuation of the ultrasound waves using signal or image processing after the data set has been captured, as compared to “physical attenuation” in which the ultrasound waves are attenuated during propagation by virtue of their interaction with the substance they travel through, which depends upon the physical properties of the substance.

In other examples, the second ultrasound data set may be obtained by recording a new data set using an ultrasound probe. In such examples the method may comprise adjusting imaging parameters of the ultrasound imaging system based on the signal or image processing of the first ultrasound data set. In a specific example the method may comprise reducing the transmit amplitude of the ultrasound waves (i.e., compared to the transmit amplitudes used to capture the first ultrasound data set). This may be achieved by assigning an individual electric transmit pulse to each transmit beam that forms the new data set. A change in the output voltage of a power supply is typically slew rate limited and requires some transition time. Variable acoustic output power between individual transmit beams can for example be achieved by the use of pulse width modulation techniques without changing the voltage of the power supply. The individual electric transmit pulses are preferably generated based on the signal or image processing of the first ultrasound data set. The electric transmit pulses can thus be selected to compensate for the lack of attenuation through the cavity region. In the resulting second ultrasound data set the image artifacts related to the low attenuation in the cavity are therefore suppressed effectively.

In both of the above examples the imaging system is used to suppress the artifacts, e.g., by using software to artificially attenuate the first ultrasound data set or by adjusting the parameters (e.g., individual electric transmit pulses for each transmit beam) of the ultrasound imaging system to capture new data sets having suppressed artifacts.

The method does not require surgery since the ultrasound data set can be obtained non-invasively, but the method may be of particular benefit when used alongside a surgical procedure, where high quality real-time imaging can be crucial to the success of the surgery. Thus, the method may be performed during a surgical procedure, and the ultrasound data set may be an intraoperative ultrasound data set. The surgeon carrying out the procedure can therefore use the high-quality intraoperative ultrasound images of the anatomical structure with reduced image artifacts to guide the operation, e.g., to facilitate maximal safe resection of a brain tumor from a patient's brain.

The biological tissue may comprise normal (i.e., healthy) tissue, and/or lesion tissue (e.g., tumor tissue). The acoustic attenuation of the volume of biological tissue may be a typical acoustic attenuation for soft tissues, e.g., between 0.3 to 1 dB/cm/MHz one-way.

The fluid-filled cavity within the biological tissue may be a tumor resection cavity, i.e., a cavity formed by the removal of tumor tissue from the anatomical structure. Typically, some normal tissue is also removed e.g., in order to gain access to the tumor. The fluid is provided into the cavity to obtain acoustic contact between the ultrasound probe and the tissue surrounding the cavity. Thus, the fluid within the cavity may comprise saline, water, and/or other fluids suitable for obtaining acoustic contact with the surrounding normal tissue and/or tumor tissue. The fluid is preferably a non-hazardous fluid, e.g., saline, to minimize risk to the patient by exposure to the fluid.

The fluid-filled cavity may be a naturally occurring cavity within the biological tissue, such as a blood-filled blood vessel, a heart chamber, an amniotic sac, a bladder cavity, a gall bladder cavity, a cyst, etc. Thus, the fluid within the cavity may comprise blood, amniotic fluid, urine, bile, cystic fluid, etc.

The biological tissue may comprise a plurality of such fluid-filled cavities. The method described herein may be applied to suppress artifacts related to one or more of the plurality of fluid-filled cavities.

The first ultrasound data set will typically include one or more image artifacts. The image artifact(s) may comprise one or more areas of brightening, e.g., an area of the image that is brighter relative to the other parts of the image of the same substance (but which does not depict actual differences in the structure of the substance). The artifact is typically in the area of the image behind the fluid-filled cavity, since the artifact is typically formed as a result of echoes of ultrasound waves that have travelled through the cavity without being attenuated much relative to the attenuation by other tissue substances (e.g., brain tissue). ‘Behind’ may be defined in relation to the relative position of the ultrasound probe and the cavity. Such artifacts can for example make it difficult to identify the boundary between the remaining tumor and the normal tissue in ultrasound-guided neurosurgery or they can affect the ultrasound visualization of atherosclerotic plaques, and suppressing these artifacts is therefore important.

Obtaining the first ultrasound data set of the anatomical structure may comprise receiving ultrasound image data representative of a first ultrasound image. For instance, a computer system may receive the first ultrasound data set, e.g., directly from an ultrasound imaging system or from a stored data file. The computer system may then carry out the signal or image processing. The computer system may comprise a processor configured for this purpose, i.e., to receive and process the data set. Thus, the method may be carried out using a computer system which is configured to obtain the data set and perform the signal or image processing.

Optionally, the method may comprise using an ultrasound imaging system to obtain the first ultrasound data set. For instance, the method may comprise placing an ultrasound probe of an ultrasound imaging system in a suitable location for obtaining data sets of the anatomical structure and across a region expected to include a fluid-filled cavity, and using the ultrasound probe to obtain the first ultrasound data set. The ultrasound probe may be placed in the suitable location and the ultrasound data set may be acquired during intraoperative ultrasound imaging (e.g., in brain surgery), during transcutaneous ultrasound imaging (e.g., for imaging of the carotid artery) or during endoscopic ultrasound imaging (e.g., using a transvaginal or transrectal probe). The ultrasound imaging system may then transmit the first ultrasound data set to a computer system for signal and image processing. The ultrasound imaging system may be a part of the computer system (i.e., the computer system may comprise the ultrasound imaging system) or they may be separate systems.

Typically, an ultrasound imaging system (e.g., an ultrasound scanner) comprises ultrasound front-end hardware (e.g., the ultrasound probe) connected to a computer with processing power. Since the processing for artifact suppression as described herein is typically not very computationally heavy, the processing may preferably be performed on the computer of an ultrasound scanner.

As discussed previously, obtaining the second ultrasound data set may comprise using an ultrasound imaging system to obtain the second ultrasound data set. Thus, the method may comprise placing an ultrasound probe of an ultrasound imaging system in a suitable location for obtaining images of the anatomical structure and across a region expected to include a fluid-filled cavity, and using the ultrasound probe to obtain the second ultrasound data set.

The region of interest of the first ultrasound data set may be automatically or manually defined. The region of interest preferably includes the image artifact and the attenuation is preferably applied to at least the region of interest so that the attenuation is applied to the artifact to reduce or suppress it. The region of interest is preferably a region which includes the area behind the fluid-filled cavity, since this is where the image artifact typically occurs as a result of low attenuation through the cavity. The region of interest may comprise radio frequency (RF) or in phase and quadrature (IQ) signals.

The attenuation (e.g., artificially or via a change in the electric transmit pulses) is introduced to suppress the artifact arising due to the low attenuation cavity. For intraoperative imaging of surgical resection procedures, the goal is thus to obtain the same ultrasound image quality when imaging is performed during (and especially towards the end of) the resection as at the start of the procedure (i.e., before the cavity is created). For imaging of structures in relation to naturally-occurring cavities, the goal is to strongly suppress the image artifacts generated by these cavities.

In examples in which the first ultrasound data set is attenuated to obtain the second ultrasound data set, the attenuation may be implemented in different ways.

Acoustic attenuation is an example of a filter where all frequency components in the acoustic pulse are attenuated but higher frequency components are attenuated more than lower frequency components. In a preferred example, the attenuation comprises applying such a filter to the region of interest, to mimic the physical acoustic attenuation, and the attenuation coefficient has a frequency dependence, for example as indicated in Eq. (1) below. The filter may modify radio frequency (RF) or in phase and quadrature (IQ) signals from the region of interest.

In some examples, the filter may be generated using a mathematical model for the acoustic attenuation. The region of interest in the ultrasound data set may then be modified according to the model to compensate for the lack of attenuation through the actual fluid-filled cavity.

This method may involve generating an artificial acoustic attenuation coefficient for the fluid-filled cavity. The artificial acoustic attenuation coefficient is preferably chosen to be higher than the actual acoustic attenuation coefficient of the fluid-filled cavity. This represents the cavity being filled with a substance having a higher attenuation coefficient than the attenuation coefficient of the actual fluid in the cavity. For instance, this may represent an original saline-filled resection cavity being filled with a different fluid having a higher attenuation coefficient than saline, or it may represent the original resection cavity being filled with tumor tissue or other soft tissue to mirror the pre-resection state of the anatomical structure (i.e., the anatomical structure before the tumor tissue was resected and the cavity was formed). The artificial attenuation coefficient may therefore be based on the standard acoustic attenuation in soft tissue, which may typically be between 0.3 to 1 dB/cm/MHz one-way.

The artificial acoustic attenuation coefficient may be generated by selecting values of the parameters a and b in the equation:

$$α ( f ) = a {f}^{b} , \qquad ( 1 )$$

where α is the attenuation coefficient in units of decibel (dB) per length, and f is frequency of the wave. This corresponds to the established attenuation coefficient for soft tissues, which has been found to follow a power law in frequency. As an example, a may be set as equal to 0.5 dB/cm/MHz one-way and b may be set as equal to 1. The effect of this will be the same as filling the cavity (resection cavity or naturally-occurring cavity such as for example a cyst, a blood vessel or a bladder) with a physical fluid with a constant acoustic attenuation of 0.5 dB/cm/MHz one-way and a linear frequency dependence.

A model of the attenuation of an ultrasound wave through the fluid-filled cavity having the artificial acoustic attenuation coefficient may then be created. The model may simulate how an ultrasound wave travelling through the fluid-filled cavity at a higher acoustic attenuation would behave, to determine by how much that wave would be attenuated.

The model may be based on the equation for attenuation of a plane wave:

$${I}_{z} = {I}_{0} {e}^{- 2 μ ( f ) z} , \qquad ( 2 )$$

where/o is the incident intensity, I_{z} is the intensity at depth z, f is frequency and μ is the attenuation coefficient of the tissue in units of Neper per length and is related to the attenuation coefficient α in Equation (1).

In another example, a constant value for the attenuation coefficient in Equation (1) may be used for a given region of interest, meaning that all frequency components in the signal are attenuated by the same amount. Even with this simple implementation, the attenuation is proportional to the total distance z travelled through the fluid-filled cavity and the amount of attenuation will typically vary between individual data points and/or scan lines in the data set.

Herein, we define segmentation as categorization of ultrasound data points into at least two classes, for instance representing tissue and fluid.

Creation of the model may include performing segmentation on the ultrasound data set. Segmentation may identify borders of the regions with a deviation in acoustic attenuation large enough to cause image artifacts. Preferably, the segmentation may identify boundary features that are representative of the boundaries of at least the fluid-filled cavity, because the fluid-filled cavity is the region of relatively low attenuation which typically causes the image artifact. The segmentation may involve one or more techniques such as thresholding and/or edge detection. The segmentation process may preferably be performed automatically, or the segmentation may be performed semi-automatically or manually e.g., in situations where a good segmentation cannot be obtained automatically.

Creating the model may further comprise determining a distance travelled through the cavity by an ultrasound pulse. For instance, for each data point or scan line in the data set, the one-way distance travelled through the fluid-filled cavity by the ultrasound pulse may be obtained from the segmented data set, using the identified boundary features. The generated artificial acoustic attenuation coefficient may then be combined with the total distance travelled through the fluid-filled cavity for each data point or scan line (which corresponds to z in equation 2). Thus, the model may simulate the attenuation of a wave through the fluid-filled cavity, if the fluid-filled cavity comprised a different material having a higher attenuation coefficient.

The filter may then be generated based on the model, and applied to the region of interest. This attenuates the region of interest (and thus suppresses the image artifact in the region of interest) so that the resulting ultrasound data set corresponds to an original ultrasound data set obtained before the fluid-filled cavity was created. For intraoperative applications, data sets acquired before resection or intervention has begun may be referred to as pre-data sets.

For soft tissue imaging and using equation (1) for the artificial attenuation coefficient, the parameter space for a may be from 0.3 to 1 dB/cm/MHz one-way, and for b it may be from 1 to 2 but other values can also be used.

In other examples, the filter may be generated based on data sets obtained before and after the fluid-filled cavity has formed, e.g., before and after tumor resection has begun.

In such examples, an ultrasound pre-data set of the anatomical structure may be obtained before the cavity has formed (e.g., before any tumor or normal tissue has been resected), and the first ultrasound data set may be obtained after the cavity has formed. The ultrasound pre-data set may preferably be a data set of the tumor to be resected and surrounding normal tissue. The first ultrasound data set may then be obtained when required during surgery and at the end of surgery, showing the surgical cavity created by resection and the surrounding tissue. The first ultrasound data set is then the ultrasound data set that comprises the image artifact and to which the filter is applied to reduce the artifact as described herein. Preferably this method is used with a pre-data set obtained before the formation of a cavity but the method can also be applied using a pre-data set that is obtained after a cavity has formed (but earlier in the surgical procedure/cavity formation process than the obtaining of the first ultrasound data set).

The ultrasound pre-data set and the first ultrasound data set are preferably obtained using an ultrasound probe which is in substantially the same position relative to the anatomical structure. This allows more accurate comparison of corresponding regions of the pre- and post-data sets. With the current generation of navigation systems using tracking (e.g., optical tracking) of surgical instruments and ultrasound probes, the position of the ultrasound probe relative to the anatomical structure can be kept substantially fixed for the repeated imaging required to obtain the pre- and first data sets and to generally follow the progress of the surgical resection.

The filter for attenuating the region of interest may then be created based on certain signal or image characteristics in a region of interest of the first ultrasound data set (i.e., the region including the image artifact) and a region of interest of the ultrasound pre-data set (which preferably does not include an image artifact since no low-attenuation cavity has yet been formed). Relevant signal or image characteristics to be used in the design of the filter may be obtained using mathematical transforms such as the Fourier transform or wavelet transforms or any other mathematical transform or signal decomposition. Power spectral density as a basis for the filter is briefly described below but this is to be understood as an example only.

Optionally, obtaining the filter may comprise estimating a power spectral density of each region of interest. For instance, for a given segment (e.g., scan line segment) in a region of interest, an estimate of the power spectral density may be obtained by applying a window function (e.g., a Hann window) and a discrete Fourier transform.

The power spectrum can be prone to a relatively large variance caused by statistical fluctuations in the echo signal from random media such as soft tissues. This variance may be reduced by averaging multiple local power spectra of partially overlapping segments of the region of interest in the range (i.e., depth) direction. Additionally and/or alternatively, to reduce the variance, the method may include averaging multiple local power spectra in the lateral direction between different points or scan lines within the region of interest. A high degree of lateral averaging will typically result in reduced variance for the power spectrum estimate and in a filter with reduced sensitivity for lesion heterogeneity in the lateral direction. Similarly, little lateral averaging will typically result in increased variance for the power spectrum estimate and in a filter with increased sensitivity for lesion heterogeneity in the lateral direction. A practical trade-off may therefore be established to optimize power spectrum variance and filter sensitivity for lesion heterogeneity.

Different techniques for estimation of the power spectral density may be used. These techniques are typically divided into non-parametric and parametric methods. According to the Wiener-Khinchin theorem, the power spectral density of a stationary (wide-sense) random process is equal to the Fourier transform of the autocorrelation function of said random process. Non-parametric methods are usually based on the squared modulus of the discrete Fourier transform (the periodogram) and usually involve averaging of multiple segments where said segments often are overlapping (e.g., Welch's method). Parametric methods for estimation of the power spectral density are often based on the autoregressive model (AR), the moving-average model (MA) or a combination of these. Any of these methods or a combination may be used to obtain the estimations.

Once the power spectral densities for the corresponding regions of interest of the pre- and first data sets have been estimated, the power spectral densities may be compared. The filter for attenuating the first data set may then be generated based on the estimated power spectral densities for corresponding regions of interest of the pre- and first data sets.

The filter may be generated using one of these example methods or a combination, and/or other methods may be used.

Once the filter has been generated, the filter may be applied to the region of interest including the image artifact. The filter modifies the data set and attenuates the signals, resulting in a final (second) data set in which the image artifact has been reduced.

The artificial attenuation may also be based on a combination of the two above-described methods (i.e., the artificial attenuation coefficient method and the pre- and post-data set comparison method) and/or with other methods.

In examples where the second data set is obtained by capturing a new data set with adjusted imaging parameters, rather than modifying the existing data set, the method may initially be based on the first and/or second above-described methods. For instance the method may include some initial processing (e.g., for cavity detection and segmentation) which may include features or steps of the first and/or second methods. Then, based on that processing, the computer software may determine how to alter the imaging parameters, e.g., different electric transmit pulses for each transmit beam, to achieve a better image and suppress the image artifact. A new data set can then be captured by the ultrasound probe e.g., using transmit beams having the adjusted electric transmit pulses.

The reduction of the image artifact may be understood as reducing the appearance of the artifact or suppressing the artifact. Preferably, the image artifact is removed completely so that the processed ultrasound image is an accurate representation of the anatomical structure and cavity.

Thus, with the present method, the image artifact is reduced by using post-processing software to process the first data set and obtain a second data set in which the cavity region has been compensated for, e.g., by artificially attenuating the ultrasound signals of the first data set or adjusting imaging parameters such as the electric transmit pulses of the ultrasound imaging system.

For intraoperative applications in particular, the benefits of using a software-based method compared to physically attenuating the signals (e.g., using a synthetic fluid) include that any concerns related to toxicity and the need for cleaning of the cavity after using a synthetic fluid will be avoided. Furthermore, the removed tumor tissue will be a heterogeneous mass with an unknown attenuation that can vary greatly between individual patients. In respect of brain tissue it has been shown that the attenuation in grey matter is lower than in white matter, and little is known about the attenuation in brain tumors. The acoustic attenuation will also typically vary within different regions of the ultrasound image because the removed tissue mass is not homogeneous for a given patient. Hence, a liquid with a constant attenuation will only to some extent lessen the formation of such image artifacts, and brings many other hazards. The present software-based method avoids such disadvantages.

As described previously, the method may be partially or completely carried out using a computer system and/or software.

Viewed from a second aspect, the present invention provides a computer program product comprising instructions that, when executed, will configure a computer system to carry out the method of the first aspect.

Thus, the computer program product of this aspect may configure the computer system to: obtain a first data set of the anatomical structure including the biological tissue and the fluid-filled cavity, the first data set comprising a region of interest; process the first data set using computer implemented signal or image processing techniques, wherein the processing includes compensating for the lower acoustic attenuation in the fluid-filled cavity; and obtain a second data set of the anatomical structure including the biological tissue and the fluid-filled cavity, wherein the second data set comprises said region of interest and where image artifacts related to the lower acoustic attenuation of said fluid-filled cavity are suppressed relative to said first data set.

The instructions may configure the computer system to carry out further features of the method as set out above in relation to the first aspect.

The computer system may comprise an ultrasound imaging device. The instructions may configure the computer system to use an ultrasound probe of the ultrasound imaging device to carry out various features of the method, e.g., to obtain the first and/or second data set of the anatomical structure including the biological tissue and a fluid-filled cavity.

Viewed from a third aspect, the present invention provides an apparatus for reducing an image artifact in a data set of an anatomical structure, the anatomical structure comprising a volume of biological tissue having a first acoustic attenuation and a fluid-filled cavity having a second acoustic attenuation lower than the first acoustic attenuation, the apparatus comprising a computer system configured to carry out the method of the first aspect.

Thus, the computer system of this aspect may be configured to: obtain a first data set of the anatomical structure including the biological tissue and a fluid-filled cavity, the first data set comprising a region of interest; process the first data set using computer implemented signal or image processing techniques, wherein the processing includes compensating for the lower acoustic attenuation in said fluid-filled cavity; and obtain a second data set of the anatomical structure including the biological tissue and a fluid-filled cavity, wherein the second data set comprises said region of interest and where image artifacts related to the lower acoustic attenuation of said fluid-filled cavity are suppressed relative to said first data set.

The computer system may optionally be configured to carry out further features of the method as set out above.

The apparatus may further comprise an ultrasound imaging device including an ultrasound probe. The ultrasound imaging device may be configured to carry out further features of the method, e.g., obtaining the first and/or second data set.

Exemplary imaging applications with which the present method and apparatus may be used include transcutaneous imaging, endoscopic imaging using an endocavity probe, intraoperative imaging of the brain or other organs, neonatal imaging of the brain, imaging of atherosclerotic plaques (e.g., at a distal artery wall), imaging of a cyst, imaging of the bladder, myocardial imaging, fetal imaging, imaging of the gall bladder and the part of the liver located behind the gall bladder relative to the ultrasound probe.

The methods and apparatuses described herein may be particularly useful when the anatomical structure comprises a brain, due to the previously mentioned difficulties in neurosurgical imaging and the importance of maximizing brain tumor resection whilst minimizing healthy brain tissue removal.

Therefore, provided herein is a method for suppressing image artifacts in an ultrasound data set of a brain, the brain comprising a volume of biological tissue having a first acoustic attenuation and a fluid-filled cavity having a second acoustic attenuation lower than the first acoustic attenuation, the method comprising: obtaining a first data set of the brain including the biological tissue and the fluid-filled cavity, the first data set comprising a region of interest; processing the first data set using computer implemented signal or image processing techniques, wherein the processing includes compensating for the lower acoustic attenuation in the fluid-filled cavity; and obtaining a second data set of the brain including the biological tissue and the fluid-filled cavity, wherein the second data set comprises said region of interest and where image artifacts related to the lower acoustic attenuation of said fluid-filled cavity are suppressed relative to said first data set.

Certain embodiments will now be described by way of example only and with reference to the accompanying drawings, in which:

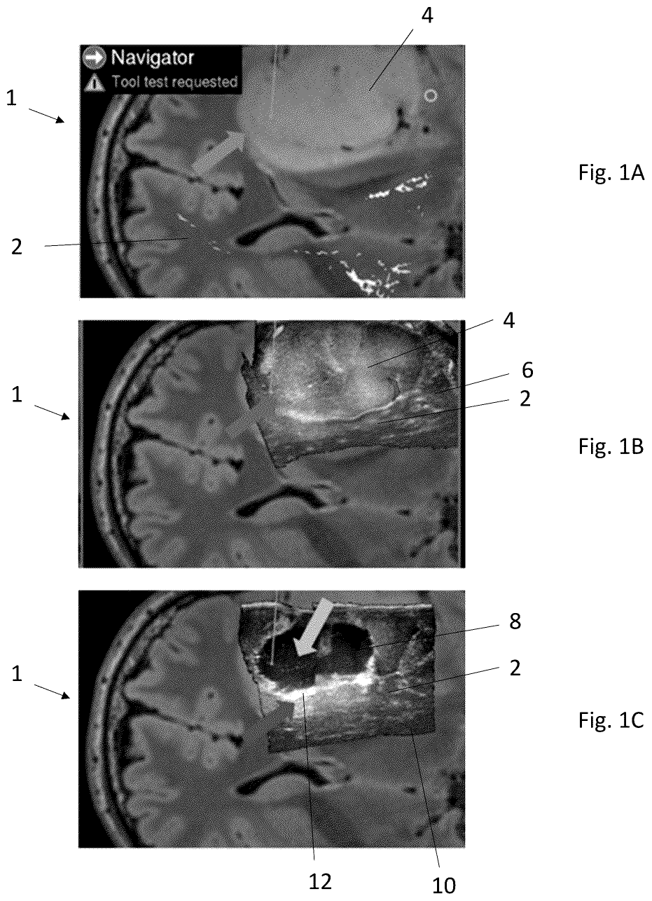

FIG. 1A shows a pre-operative magnetic resonance (MR) data set of a brain including a brain tumor to be resected;

FIG. 1B shows a navigated intraoperative ultrasound data set of the brain tumor superimposed on the pre-operative MR data set of FIG. 1A before the brain tumor resection has started;

FIG. 1C shows a navigated intraoperative ultrasound data set of a resection cavity towards the end of the brain tumor resection superimposed on the pre-operative MR data set of FIG. 1A;

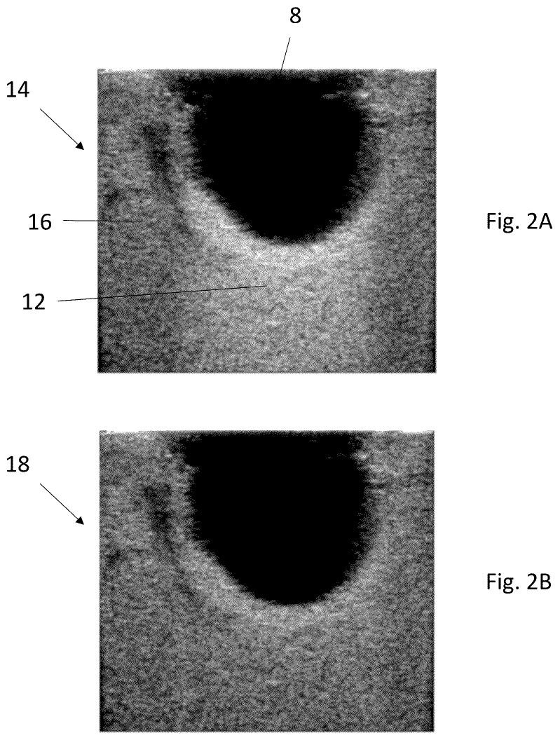

FIG. 2A shows an example ultrasound data set from a tissue-mimicking phantom before processing, the data set including a fluid-filled cavity and an image artifact; and

FIG. 2B shows the example ultrasound data set of FIG. 2A after processing for artifact reduction.

Ultrasound waves are elastic pressure waves involving a cyclic exchange of energy between a kinetic energy form related to vibration velocity and a potential energy form related to material compression. Soft tissue is a matrix of bio-molecules that typically is filled with 60-70% of water where the extracellular matrix is a network of macromolecules providing structural and biochemical support to the surrounding cells. When ultrasound waves are incident on soft tissue, the solid constituents of the extracellular matrix and the surrounding cells provide inhomogeneities in mass density and compressibility responsible for scattering (i.e., echoes) of the ultrasound waves. For the scattering to be effective, the dimension of the inhomogeneities must approach the ultrasound wavelength. These tissue inhomogeneities will also give rise to acoustic absorption which is the conversion of acoustic energy to heat.

Ultrasound attenuation is defined as the sum of acoustic scattering and acoustic absorption. In soft tissues, scattering usually contributes less than 10% (typically around 2%) to the total attenuation. A homogeneous medium like water or saline will not provide scattering of ultrasound, and acoustic absorption will be negligible compared to soft tissues.

The attenuation of a plane wave, where effects due to diffraction are removed, can be modelled according to equation 2, described above and reproduced below:

$${I}_{z} = {I}_{0} {e}^{- 2 μ ( f ) z} , \qquad ( 2 )$$

where I₀ is the incident intensity, I_{z} is the intensity at depth z, f is frequency and μ is the attenuation coefficient of the tissue in units of Neper per length. The unit of Neper is usually defined in terms of ratios of field quantities such as acoustic pressure p

$${p}_{z} = {p}_{0} {e}^{- μ ( f ) z} , \qquad ( 3 )$$

hence the factor 2 in equation 2. The unit of decibel (dB) per length, α, is more widely used and is obtained by:

$$α ( f ) = 20 ( {log}_{10} e ) μ ( f ) \approx 8.6 \; 86 μ ( f ) \qquad ( 4 )$$

From measurements of various soft tissues it has been established that the attenuation coefficient follows a power law in frequency, as shown in equation 1 above and reproduced below:

$$α ( f ) = a {f}^{b} , \qquad ( 1 )$$

where the parameters a and b depend upon the characteristics of the soft tissue. The value of b is typically in the range 1-2 and often close to unity. The attenuation in soft tissue typically approximately 0.5 dB/cm/MHz one-way.

During surgery, intra-operative ultrasound imaging can be used to augment magnetic resonance (MR) images taken pre-operatively. A conventional method for intraoperative imaging during a neurosurgical procedure is illustrated in FIGS. 1A-1C.

In FIG. 1A, an MR data set 1 of a patient's brain 2 including a brain tumor 4 is shown. This data set 1 was taken pre-operatively to identify the tumor 4 to be resected via surgery.

To guide the surgeon in the tumor resection, a combination of the pre-operative MR data set 1 and updated ultrasound data sets obtained intraoperatively were used. FIG. 1B shows the pre-operative MR data set 1 and a navigated intraoperative ultrasound data set 6 taken before the resection has started, the ultrasound data set 6 being superimposed onto the MR data set 1. The ultrasound data set 6 shows the tumor 4 to be resected.

As shown in FIG. 1C, during surgery, a cavity 8 is created as tumor tissue is surgically removed. A second ultrasound data set 10 is obtained which includes the cavity 8. When ultrasound is used as a tool to identify residual tumor mass, this cavity 8 is typically filled with saline to provide the required acoustic contact between the ultrasound probe and the tissue. When the propagation medium between the ultrasound probe and the residual tumor mass partly or completely comprises saline, an image artifact 12 is created due to the fact that the acoustic attenuation for saline is very low. The artifact 12 is the region behind the cavity 8 which is excessively bright due to the lack of ultrasound wave attenuation through the cavity 8. This artifact 12 can make it difficult for the surgeon to identify the boundaries of the tumor 4, and hence make it difficult to achieve maximal tumor resection whilst not damaging healthy brain tissue 2.

The method and apparatus described herein may be used to process such ultrasound data sets 10 to reduce the artifacts or the appearance of the artifacts, thereby improving the quality of the ultrasound images.

The proposed method is useful for ultrasound imaging of anatomical structures in relation to both naturally occurring fluid-filled cavities and resection cavities and the method proceeds as follows. An ultrasound probe is placed in a suitable location for obtaining data sets of the anatomical structures of interest. FIG. 2A shows an example data set 14 obtained by an ultrasound probe. The data set shows a volume of tissue-mimicking substance 16 (e.g., representing brain tissue, bladder tissue, liver parenchyma, etc.) in which a cavity 8 has been created (e.g., to mimic the cavity created after resection of tumor mass, the cavity of a urinary bladder, the gall bladder, the lumen of an artery, etc.) and filled with degassed water. Relative to the image, the ultrasound probe was located at the top and pointing downwards. An image artifact 12 is present in the form of a brightened region behind the cavity 8 (with “behind” being defined relative to the positions of the ultrasound probe and the cavity 8). This artifact 12 has formed because the acoustic attenuation of the fluid-filled cavity 8 is relatively low compared to the surrounding tissue-mimicking substance 16.

The data set 14 is then processed using computer implemented signal or image processing techniques. Three example methods are discussed herein. The data sets used in the examples are based on the fundamental (i.e., transmitted) frequency band but the methods for artifact suppression are applicable also in situations where harmonic imaging techniques are applied and where the resulting data sets for example are based on the received second harmonic frequency band.

### Example 1: Filter Based on a Mathematical Model

A first example processing method involves using a mathematical model to model the attenuation and create a filter based on the model to compensate for the low attenuation caused by the fluid-filled cavity 8. The model is based on equations (1) and (2) defined above.

An assumption regarding the acoustic attenuation of the resected tissue is made. Both a constant acoustic attenuation (i.e., without any variations between scan lines in the lateral direction) and a variable acoustic attenuation may be used.

The first step is to perform segmentation to find the borders of the fluid-filled cavity region 8 and the tissue region 16 in FIG. 2A. Then, for each scan line in the data set 14, the one-way distance travelled through the fluid-filled cavity region 8 by the ultrasound pulse are obtained based on the segmented data set 14.

Predefined values of the parameters a and b in equation (1) are then used to compensate for the deviation in acoustic attenuation between regions. In this example, a is set as equal to 0.5 dB/cm/MHz one-way, and b is set as equal to 1.

For each scan line, this is then combined with the total distance travelled through the fluid-filled cavity region 8 (which corresponds to z in equation (2)).

This results in a filter that attenuates the signals to which it is applied.

The image artifact 12, which is an inherent part of the signals from the region of interest behind the fluid-filled cavity 8, is then suppressed by the resulting filters by applying the filters to the region of interest. Thus, the ultrasound data set is attenuated artificially.

The resulting data set 18 is shown in FIG. 2B. It can be seen that the bright artifact 12 behind the fluid-filled cavity 8 in FIG. 2A has been attenuated using the post-processing method. The effect of this is the same as filling the cavity 8 with a physical fluid having a constant acoustic attenuation of 0.5 dB/cm/MHz one-way and a linear frequency dependence.

The above steps may be performed on individual data points in the data set rather than scan lines. For example, for each data point in the data set 14, the one-way distance travelled through the fluid-filled cavity region 8 by the ultrasound pulse may be obtained based on the segmented data set 14, and for each data point the predefined values of the parameters a and b may then be combined with the total distance travelled through the fluid-filled cavity region 8 to create the filter.

### Example 2: Filter Based on Pre- and Post-Data Sets

A second example processing method involves compensating for the deviation in acoustic attenuation by designing a filter based on certain signal or image characteristics from pre- and post-data sets (i.e., data sets taken before and during/after resection). This method will be discussed with reference to FIGS. 1B and 1C and is only relevant for application where the cavity is created by an intervention (i.e., in intraoperative imaging).

Before any surgical cavity 8 has been created, a pre-data set 6 displaying the tumor 4 with surrounding normal tissue 2 is obtained, e.g., as shown in FIG. 1B. Then, when required during surgery and at the end of surgery, one or more post-data sets 10 are obtained which include the surgical cavity 8 and an image artifact 12, e.g., as shown in FIG. 1C. This method is preferably applied using a pre-data set without artifacts due to surgical cavities but can also be applied using a pre-data set obtained after the surgical intervention has started in the presence of a cavity. The method is further exemplified below using the power spectral density as a basis for the filter but it is emphasized that the method can be applied using e.g., the complex Fourier spectrum or wavelet transforms or any other mathematical transform or signal decomposition rather than the power spectral density.

For a given post-data set 10, the attenuation compensation in this example is obtained by a filter based on the power spectral density of a region of interest including the image artifact 12 in the post-image 10 and the power spectral density of substantially the same region of interest in the pre-data set 6.

For a given segment (e.g., scan line segment) in a region of interest, an estimate of the power spectral density may be obtained by applying a window function (e.g., a Hann window) and a discrete Fourier transform. Averaging of multiple local power spectra can be performed to reduce the variance caused by statistical fluctuations in the echo signal from random media such as soft tissues.

The filter is then based on the estimated power spectral densities in the pre- and post-data sets, for example by comparing power spectral densities within regions of interest in the pre- and post-data sets. Finally, the filter is applied to the region of interest of the post-data set 10, and the image artifact 12 is thereby suppressed.

An advantage of this method as compared to the first example method is that no assumptions about the acoustic attenuation are required. This method will benefit from a tracking system with high accuracy to enable the ultrasound probe position and orientation relative to the region of interest to be substantially the same for the pre- and post-data sets 6, 10. Another advantage is that this method allows implementation of a filter that varies with respect to the lateral direction to account for heterogeneity of the resected lesion 4.

A combination of methods 1 and 2 could be used to attenuate the ultrasound data sets and suppress the image artifacts.

### Example 3: Individual Electric Transmit Pulses

A third example method involves processing the data set to identify and segment the fluid-filled cavity, e.g., using techniques as described in methods 1 and/or 2. Then, a new ultrasound data set is obtained where compensation for the low attenuation of the cavity is achieved by assigning an individual electric transmit pulse to each of the transmit beams that form the new data set. Variable acoustic output power between individual transmit beams can for example be achieved using pulse width modulation techniques. The individual electric transmit pulses are based on the signal and image processing from method 1 and/or method 2.

1. A method for suppressing image artifacts in an ultrasound data set of an anatomical structure, the anatomical structure comprising a volume of biological tissue having a first acoustic attenuation and a fluid-filled cavity having a second acoustic attenuation lower than the first acoustic attenuation, the method comprising:
obtaining a first ultrasound data set of the anatomical structure including the biological tissue and the fluid-filled cavity, the first ultrasound data set comprising a region of interest;
processing the first ultrasound data set using computer implemented signal or image processing techniques, wherein the processing includes compensating for the lower acoustic attenuation in the fluid-filled cavity; and
obtaining a second ultrasound data set of the anatomical structure including the biological tissue and the fluid-filled cavity, wherein the second ultrasound data set comprises said region of interest and where image artifacts related to the lower acoustic attenuation of said fluid-filled cavity are suppressed relative to said first ultrasound data set.

2. The method of claim 1, wherein obtaining the second ultrasound data set comprises attenuating at least part of the first ultrasound data set.

3. The method of claim 2, wherein the processing comprises generating a filter, and wherein the attenuation comprises applying the filter to the first ultrasound data set.

4. The method of claim 3, wherein the processing comprises:
generating an artificial acoustic attenuation coefficient of the fluid-filled cavity, wherein the artificial acoustic attenuation coefficient is higher than an actual acoustic attenuation coefficient of the fluid-filled cavity;
creating a model of the attenuation of an ultrasound wave travelling through the fluid-filled cavity having the artificial acoustic attenuation coefficient; and
generating the filter based on the model.

5. The method of claim 4, wherein the artificial acoustic attenuation coefficient is based on acoustic attenuation of soft tissue.

6. The method of claim 4, wherein the artificial attenuation coefficient is generated by selecting values of the parameters a and b in the equation
$$α ( f ) = a {f}^{b} ,$$
where α is the artificial attenuation coefficient and f is frequency of the ultrasound wave.

7. The method of claim 4, wherein the artificial attenuation coefficient has a constant value which does not vary with frequency.

8. The method of claim 4, wherein creating the model comprises performing segmentation on the first ultrasound data set to identify boundary features that are representative of the boundaries of at least the fluid-filled cavity; and
wherein creating the model optionally comprises, for each data point or scan line in the first ultrasound data set, using the identified boundary features to determine a distance travelled through the fluid-filled cavity by the ultrasound wave.

9. The method of claim 1, wherein the first ultrasound data set is obtained after the cavity has formed, and wherein the method further comprises obtaining an ultrasound pre-data set of the anatomical structure before the cavity has formed or earlier in the cavity formation process compared to the first ultrasound data set, the first ultrasound data set and the ultrasound pre-data set being obtained using an ultrasound probe which is in substantially the same position relative to the anatomical structure.

10. The method of claim 9, wherein processing the first ultrasound data set comprises:
finding signal or image characteristics from the region of interest of the first ultrasound data set and a corresponding region of interest of the ultrasound pre-data set; and
generating the filter based on the signal or image characteristics from said ultrasound pre-data set and first ultrasound data set.

11. The method of claim 10, wherein finding signal or image characteristics from the regions of interest comprises estimating a power spectral density or a complex Fourier spectrum or applying a wavelet transform or any other mathematical transform or signal decomposition of each region of interest.

12. The method of claim 11, wherein finding the signal or image characteristics of each region of interest comprises, for each point or scan line in the respective region of interest, applying a window function.

13. The method of claim 11, wherein finding the signal or image characteristics of each region of interest comprises averaging multiple local signal or image characteristics of partially overlapping segments of the respective region of interest in the range direction, and/or averaging multiple local signal or image characteristics in the lateral direction between different points or scan lines within the respective region of interest.

14. (canceled)

15. The method of claim 1, wherein obtaining the first and/or second ultrasound data set comprises placing an ultrasound probe in a suitable location for obtaining signals of the anatomical structure and across a region expected to include the fluid-filled cavity, and using the ultrasound probe to obtain the first and/or second ultrasound data set.

16. The method of 15, wherein using the ultrasound probe to obtain the second ultrasound data set comprises assigning an individual electric transmit pulse to each transmit beam forming the second ultrasound data set; optionally
wherein the individual electric transmit pulses are determined based on the signal or image processing of the first ultrasound data set and/or wherein pulse width modulation techniques are used to modulate the acoustic output power of the ultrasound probe.

17. (canceled)

18. The method of claim 15, wherein the ultrasound probe is placed in the suitable location during a surgical procedure such that the ultrasound data set is an intraoperative ultrasound data set.

19. The method of claim 1, wherein the anatomical structure comprises a brain, a bladder, a gall bladder or liver parenchyma behind the gall bladder, a foetus, a heart, an atherosclerotic plaque or the anatomical structures behind a large blood vessel.

20. A computer programme product comprising instructions that, when executed, will configure a computer system to carry out the method of claim 1.

21. (canceled)

22. (canceled)

23. An apparatus for suppressing image artifacts in an ultrasound data set of an anatomical structure, the anatomical structure comprising a volume of biological tissue having a first acoustic attenuation and a fluid-filled cavity having a second acoustic attenuation lower than the first acoustic attenuation, the apparatus comprising:
a computer system configured to carry out the method of claim 1.

24. The apparatus of claim 23, wherein the computer system comprises an ultrasound imaging device including an ultrasound probe configured for obtaining the first and/or second ultrasound data set.

25. (canceled)
